SEQUENCE LISTING (2 sequences)

SEQ ID NO: 1 - protein, 9 aa, Homo sapiens
  MOD_RES (9)..(9): AMIDATION
CYIQNCPLG

SEQ ID NO: 2 - protein, 10 aa, Artificial sequence
  Oxytocin fragments and variants
  MISC_FEATURE (1)..(1): X1 can be Cys or nothing
  MISC_FEATURE (2)..(2): X2 can be Tyr, Phe or nothing
  MISC_FEATURE (3)..(3): X3 can be Ile, Val, Hoph, Phe, Cha or nothing
  MISC_FEATURE (4)..(4): X4 can be Gln, Ser, Thr, Cit, Arg or Daba
  MISC_FEATURE (7)..(7): X5 (position 7) can be Pro or nothing
  MISC_FEATURE (8)..(8): X6 (position 8) can be Ile, Leu, nothing, Val, Hos, Daba, Thr, Arg or Cit
  MISC_FEATURE (9)..(9): X7 (position 9) can be Gly, nothing or Ala
  MOD_RES (10)..(10): AMIDATION
  MISC_FEATURE (10)..(10): X8 (position 10) can be Gly or nothing
XXXXNCXXXX

(19) United States
(12) Patent Application Publication
(10) Pub. No.: US 2022/0362384 A1
(43) Pub. Date: Nov. 17, 2022

(54) COMPOSITION FOR TREATING OR PREVENTING CLIMACTERIC DISORDERS

(71) Applicant: PEPTONIC MEDICAL AB, Växjö (SE)

(72) Inventors: Dan Markusson, Växjö (SE); Johan Inborr, Växjö (SE); Anders Carlsson, Stockholm (SE)

(73) Assignee: PEPTONIC MEDICAL AB, Växjö (SE)

(21) Appl. No.: 17/848,554

(22) Filed: Jun. 24, 2022

Related U.S. Application Data
(63) Continuation of application No. 16/617,269, filed on Nov. 26, 2019.

(30) Foreign Application Priority Data
May 30, 2017 (SE) 1750680-9

(51) Int. Cl. A61K 47/38; A61P 15/12; A61K 47/12

(52) U.S. Cl. CPC A61K 47/38; A61P 15/12; A61K 47/12

(57) ABSTRACT

The present document is directed to a pharmaceutical composition comprising at least one non-ionic cellulose ether, wherein said composition has a viscosity of 35000 cP or more, an osmolality of from about 10 to about 300 mOsmol/kg, and a pH of from about 3 to about 4. The composition may be used in the treatment and/or prevention of a climacteric disorder, wherein said climacteric disorder is a selected from the group consisting of vaginal dryness, vaginal irritation, vaginal itching, dysuria, dyspareunia, and/or vaginal bleeding during and/or after sexual intercourse and any combination thereof.

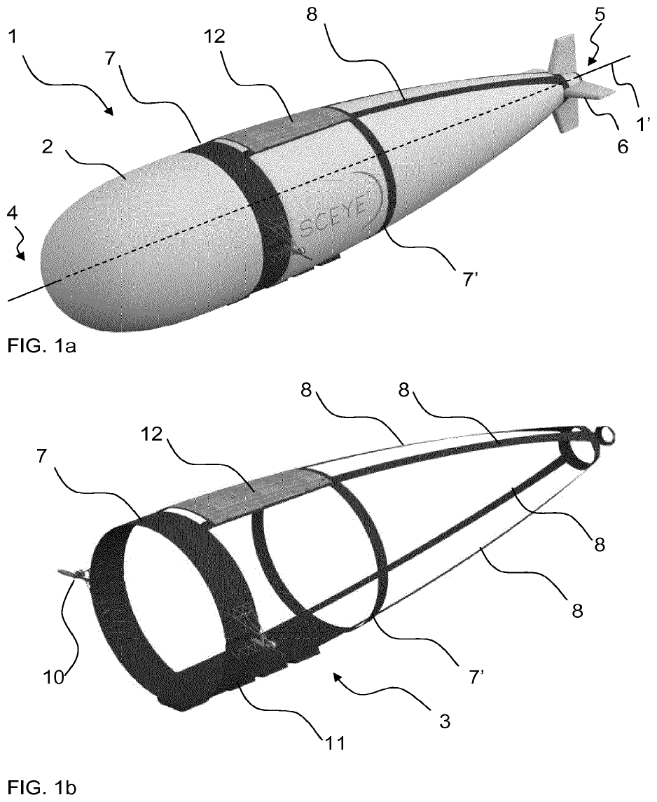

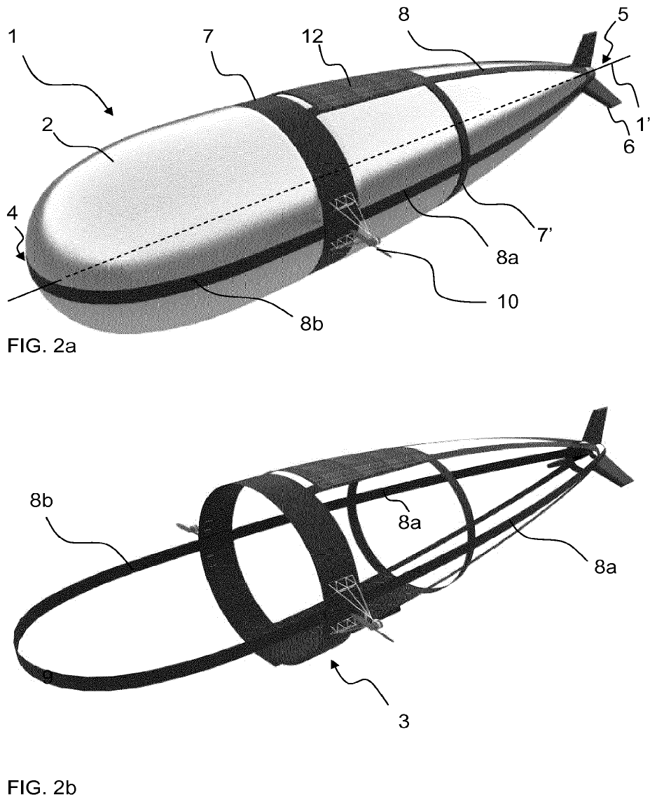

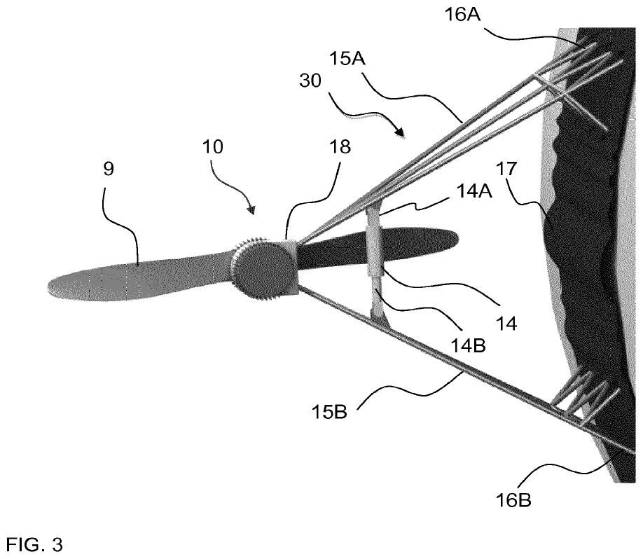

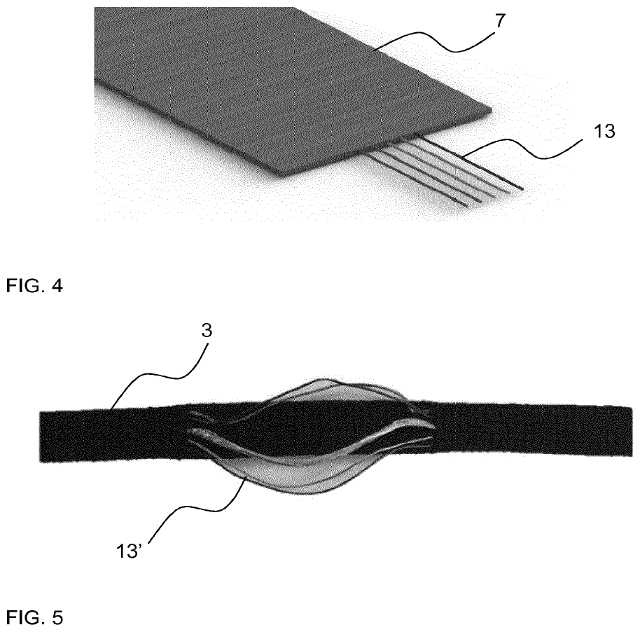

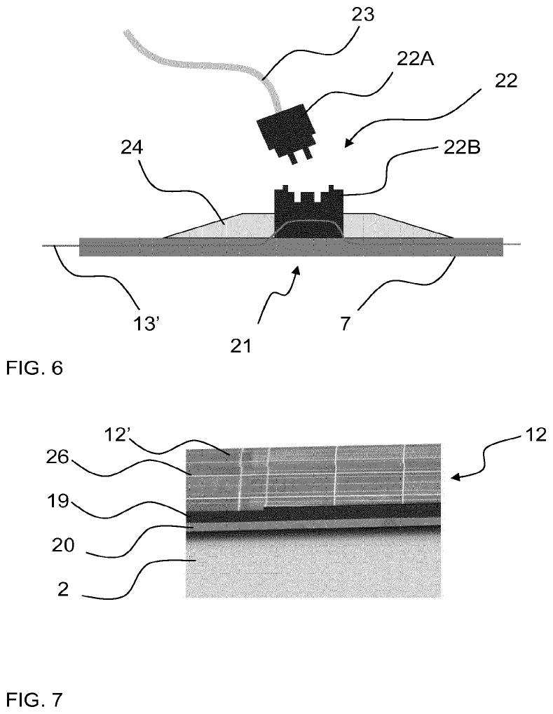

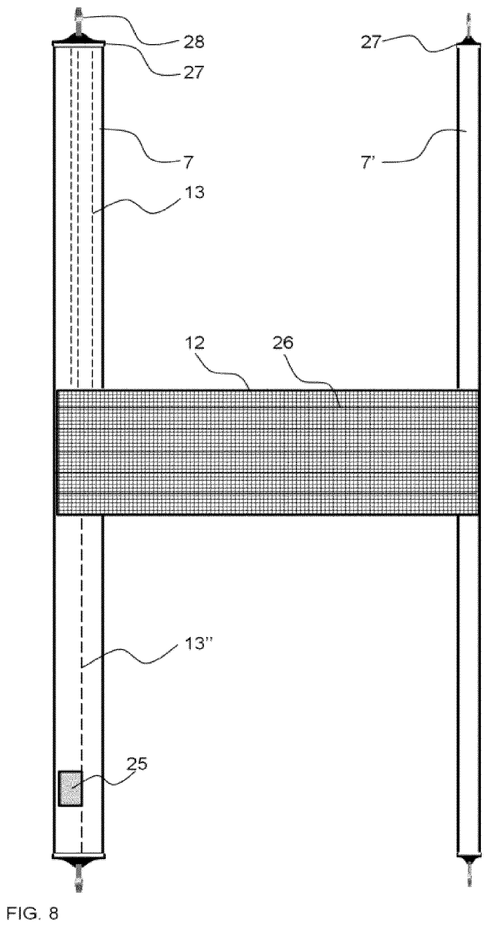

### CROSS-REFERENCE TO RELATED APPLICATIONS

This application is a Continuation of, and claims priority under 35 U.S.C. § 120 to U.S. patent application Ser. No. 16/617,269, filed Nov. 26, 2019, which was a National Stage Entry under 35 USC 371 of PCT/EP2018/063547, filed May 23, 2018, which claimed priority from Swedish patent application 1750680-9, filed May 30, 2017. The contents of these priority applications are incorporated herein by reference in their entirety. Also, the Sequence Listing filed electronically herewith is hereby incorporated by reference (File name: 123-005C_Seq_Listing; File size: 2 KB; Date recorded: Jul. 12, 2022).

### TECHNICAL FIELD

The present document is directed to a composition for use in the treatment and/or prevention of conditions associated with climacteric disorders, such as vaginal dryness, vaginal irritation, vaginal itching, dysuria, dyspareunia, and/or vaginal bleeding during and/or after sexual intercourse. The composition is a gel comprising a non-ionic cellulose ether and the treatment involves vaginal administration of the gel.

### BACKGROUND

During and after menopause women can experience several different climacteric disorders, such as vaginal dryness, vaginal irritation, vaginal itching, dysuria, dyspareunia, and/or vaginal bleeding during and/or after sexual intercourse and any combination thereof. Today, such disorders are often treated using hormone replacement therapy, such as administration of different forms and formulations of oestrogen. However, such hormone replacement therapies may be associated with side effects such as increased risk for strokes, blood clots and cancer.

Cellulose ethers are named after, and based on, cellulose which is a renewable material and the most common organic chemical compound in nature. There is a broad range of cellulose ethers available on the market, both ionic and non-ionic, for example sodium carboxymethylcellulose, hydroxyethylethylcellulose, hydroxyethylcellulose, hydroxypropylcellulose, hydroxyethylmethylcellulose and hydroxypropylmethycellulose.

Cellulose ethers are used as additives in such diverse applications as food, paint, oil recovery, paper, cosmetics, pharmaceuticals, adhesives, printing, agriculture, ceramics, textiles, detergents and building materials. Cellulose ethers improve the product quality in these applications and act as thickeners, water retention agents, suspending aids, protecting colloids, film formers or thermoplastics in such different products as dispersion paints, drilling muds, ice cream, tablet coatings, wallpaper paste and tile adhesive.

Non-ionic cellulose ethers such as methylcellulose, hydroxypropylmethylcellulose (also referred to as hypromellose) and methylhydroxyethylcellulose, are widely used in the pharmaceutical industry due to their ability to thicken, bind and retain water, as well as to emulsify and suspend particles and form films. Further information regarding non-ionic cellulose ethers can be found e.g. in WO92/09307.

An object of the present invention is to overcome or at least mitigate some of the problems associated with the prior art.

### SUMMARY OF INVENTION

The present document is directed to a pharmaceutical composition comprising at least one non-ionic cellulose ether, wherein said composition has a viscosity of 35 000 cP or more, an osmolality of from about 10 to about 300 mOsmol/kg, and a pH of from about 3 to about 4. The pharmaceutical composition may or may not comprise one or more active pharmaceutical ingredient(s).

The present document is also directed to such a pharmaceutical composition for use in the treatment and/or prevention of a climacteric disorder, wherein said climacteric disorder is a selected from the group consisting of vaginal dryness, vaginal irritation, vaginal itching, dysuria, dyspareunia, and/or vaginal bleeding during and/or after sexual intercourse and any combination thereof.

The present document is further directed to a method for treating and/or preventing a climacteric disorder, said climacteric disorder being selected from the group consisting of vaginal dryness, vaginal irritation, vaginal itching, dysuria, dyspareunia, and/or vaginal bleeding during and/or after sexual intercourse and any combination thereof, wherein said method comprises administration of a pharmaceutically effective amount of a pharmaceutical composition as defined herein.

The present document is also directed to the use of a non-ionic cellulose ether for the manufacture of a pharmaceutical composition as defined herein for the treatment and/or prevention of a climacteric disorder, wherein said climacteric disorder is a selected from the group consisting of vaginal dryness, vaginal irritation, vaginal itching, dysuria, dyspareunia, and/or vaginal bleeding during and/or after sexual intercourse and any combination thereof.

The present document is also directed to a kit of parts comprising:

(i) a pharmaceutical composition as defined herein;

(ii) a dispenser for said pharmaceutical composition, and

(iii) optionally instructions for use.

Other features and advantages of the invention will be apparent from the following detailed description, drawings, examples, and from the claims.

### DEFINITIONS

A “pH regulating agent” is any agent, such as a liquid agent, such as an aqueous liquid, which is able to regulate and/or maintain the pH of said pharmaceutical composition, wherein said pH is kept approximately in a selected range, which selected range is exemplified herein. Such a pH regulating agent can for example be a buffer, such as a citrate, lactate or phosphate buffer. A “buffer” is an ionic compound, usually a salt of a weak acid or base, added to a solution to resist changes in its acidity or alkalinity and thus stabilize its pH. A buffer solution is a solution containing such a compound. Other examples of a pH regulating agents are organic and inorganic acids and bases, such as acetic acid, citric acid, phosphoric acid, hydrochloric acid and sodium hydroxide.

The cellulose ethers used in the composition disclosed in this document are non-ionic, with alkyl and/or hydroxyalkyl groups attached to the anhydroglucose units by ether linkages, which form hydroxyalkylalkylcelluloses, wherein the alkyl groups have from 1 to 4 carbon atoms.

Representative cellulose ethers for use in the pharmaceutical compositions according to the present invention are methylcellulose (MC), hydroxyethylmethylcellulose (HEMC), hydroxypropylmethylcellulose (HPMC), hydroxyethylethylcellulose (HEEC), and hydroxypropylcellulose (HPC). These polymers have substituents that are either nonpolar (e.g. methyl) or slightly polar (e.g. hydroxyethyl), which in combination with the hydrophilic cellulose backbone provide an amphiphilic polymer.

The viscosity of the pharmaceutical composition disclosed herein was measured at 20° C. according to European Pharmacopoeia 7.0, 2.2.10, e.g. using spindle viscometer Brookfield DV-I Prime with spindle number SC4-28 at 1 rpm (revolutions per minute) unless otherwise specified. The torque value should be ≥10% for the result to be stable and reliable. The Brookfield instrument will display a warning light if the torque value is <10%. The correct performance of the instrument was regularly checked with reference standards (oils with different viscosities) supplied by Brookfield. The viscosity is given in cP (centipoise).

By “composition” is in the context of the present document intended a composition suitable for medical use. The composition may also be denoted a “medical composition” or a “pharmaceutical composition”.

The “Most Bothersome Symptom” (MBS) is in the context of the present document defined as the climacteric disorder symptom that is most bothersome to a woman, wherein the climacteric disorder selected from the group consisting of vaginal dryness, vaginal irritation, vaginal itching, dysuria, dyspareunia, and/or vaginal bleeding during and/or after sexual intercourse.

By “osmolality” is meant the concentration of an osmotic solution when measured in osmols or milliosmols per 1 kg of solvent.

By room temperature is meant a temperature of about 20-25° C.

### DETAILED DESCRIPTION

The present document is based on the surprising finding that a composition comprising at least one non-ionic cellulose ether and which composition has a viscosity of about 35 000 cP or more is effective in treating and/or preventing a climacteric disorder selected from the group consisting of vaginal dryness, vaginal irritation, vaginal itching, dysuria, dyspareunia, and/or vaginal bleeding during and/or after sexual intercourse and any combination thereof.

The present document is directed to a pharmaceutical composition comprising at least one non-ionic cellulose ether, wherein said composition has a viscosity of about 35 000 cP (1 centipoise (cP)=1 mPa s) or more, an osmolality of from about 10 to about 300 mOsmol/kg (mosmolal), and a pH of from about 3 to about 4 at room temperature. The pharmaceutical composition may or may not comprise one or more an active pharmaceutical agent.

The composition may have a viscosity of at least about 38 000, about 40 000, about 45 000, about 47 000, about 50 000, about 52 000, or about 55 000 cP. For example, the composition may have a viscosity of from about 35 000 to about 100 000, from about 38 000 to about 100 000, from about 40 000 to about 100 000, from about 45 000 to about 100 000, from about 47 000 to about 100 000, from about 50 000 to about 100 000, from about 52 000 to about 100 000 or from about 55 000 to about 100 000 cP.

The viscosity as defined in this document is determined as described above by measurement at 20° C. according to Ph. Eur. 2.2.10. The viscosity values referred to herein were measured at 1 rpm unless otherwise specified. The composition may have a viscosity of at least about 38 000, about 40 000, about 45 000, about 47 000, about 50 000, about 52 000, or about 55 000 cP after storage at room temperature for about six months. The storage stability of the composition as regards viscosity may be affected by the storage conditions. For example, storing the composition refrigerated and/or in glass containers may reduce the viscosity reduction during storage.

The composition may have an osmolality of from about 10 to about 300 mOsmol/kg, such as from about 10 to about 200 mOsmol/kg, from about 20 to about 100 mOsmol/kg, from about 30 to about 50 mOsmol/kg.

The pH of the composition disclosed herein is typically within the range of from about 3 to about 4, such as from about 3 to about 3.8, such as from about 3 to about 3.5, or from about 3 to 3.3. The pH may be regulated by the addition of a pH regulating agent to the composition. The pH regulating agent may e.g. be a buffer, such as a lactate or citrate buffer or an acid or base, such as hydrochloric acid or sodium hydroxide. The concentration of a buffer to be added to the composition may be from about 20 to about 100 mM, such as from about 25 mM to about 100 mM, or from about 25 to about 50 mM, from about 25 mM to about 75 mM, or from about 50 to about 70 mM in an aqueous solution. It should be noted that these values are not exact, meaning that they can vary slightly around the values provided. Depending on which pH is required and which buffer is used in the pharmaceutical composition, the concentration of the buffer will vary in accordance with the above.

The composition may further comprise a preservative, such as benzoic acid. When benzoic acid is used as a preservative, it may be added in an amount of approximately 0.5-1.5 mg/g pharmaceutical composition, such as about 0.6, 0.7, 0.8, 0.9, 1.0, 1.1, 1.2, 1.3, or 1.4 mg/g.

The non-ionic cellulose ether may be selected from the group consisting of methylcellulose (MC), hydroxypropylmethylcellulose (HPMC), hydroxypropylcellulose (HPC), hydroxyethylethylcellulose (HEEC) and hydroxyethylmethylcellulose (HEMC) and any combination of one or more thereof.

The amount of non-ionic cellulose ether used in the pharmaceutical composition is selected so that the desired viscosity is obtained. As is known to the person skilled in the art of pharmaceutical development, the chain length of the non-ionic cellulose ethers is one parameter that affects the viscosity obtained, with shorter chain lengths providing a lower final viscosity when a certain concentration of non-ionic cellulose ethers are used than if the same concentration of non-ionic cellulose ethers with a longer chain length are used. As is also known to the person skilled in the art of pharmaceutical development, there is always a variation in the chain lengths in every batch of non-ionic cellulose ethers, which variation can be small or large. However, it is the mean chain length that affects the viscosity.

Typically, the composition comprises from about 1 to about 5% (w/w) of non-ionic cellulose ethers, such as about 1.5, 2, 2.5, 3, 3.5, 4, or 4.5% (w/w) non-ionic cellulose ether. For instance, the composition may comprise from about 2.5 to about 3.5% (w/w) non-ionic cellulose ether. However, as mentioned above, due to the variation in chain lengths between different batches of non-ionic cellulose ethers, the actual amount of non-ionic cellulose ether must be adjusted to achieve the desired viscosity. This is however routine work for the person skilled in the art of pharmaceutical development.

It was surprisingly found that a composition comprising at least one non-ionic cellulose ether, wherein said composition has a viscosity of about 35 000 cP or more, and preferably an osmolality of from about 10 to about 300 mOsmol/kg, and a pH of from about 3 to about 4, had a medical effect on climacteric disorders, despite the lack of an active pharmaceutical ingredient in the composition.

Without wishing to be bound by theory, this may be due to the composition's hypotonic properties due to its low osmolality, which results in the composition being able to deliver water to the vaginal mucosa.

Further, the composition disclosed herein has a high viscosity which is beneficial when the composition is to be administered to the vaginal mucosa as it is easier to handle and also leads to the gel staying in the vagina after administration.

Also, the composition as defined herein has good mucoadhesive properties.

In general, mucoadhesive compositions interact with the mucus layer covering the mucosal epithelial surface, and mucin molecules and increase the residence time of the composition at the site of administration.

Mucoadhesion describes the attractive forces between a composition and mucus or mucous membrane.

There are two main stages of the mucoadhesive process, the contact stage and the consolidation stage. The contact stage involves the initial wetting that occurs between the composition and the mucous membrane. This can occur mechanically by bringing together the two surfaces.

The consolidation stage affects the residence time of the composition on the surface and is governed mainly by attractive non-covalent interactions between the two surfaces but also by differences in osmotic pressure between the composition and the mucous membrane.

A low osmotic pressure of the composition, that is a hypotonic composition, will result in a flow of water from the composition to the mucous membrane.

In addition, a composition as defined herein lacking an active pharmaceutical ingredient is non-cytotoxic. Also, as the composition comprises so few ingredients, the risk for adverse reactions against it is decreased.

The composition may or may not comprise an active pharmaceutical ingredient, such as drugs primarily delivered by intravaginal administration, including but not limited to vaginally administered estrogens and progestogens (a group of hormones including progesterone), antibacterials and antifungals to treat bacterial vaginosis and yeast infections, respectively, and oxytocin.

When the composition does not comprise a pharmaceutically active ingredient, the composition may in particular not comprise oxytocin.

The composition may further comprise oxytocin, and/or one or more fragment(s) and/or variant(s) thereof according to SEQ ID NO:2, as well as pharmaceutically acceptable salts of oxytocin or a fragment and/or variant thereof. The oxytocin and/or one or more fragment(s) and/or variant(s) thereof according to SEQ ID NO:2, is typically present in the composition so that a dose of from about 50 to about 600 IU is administered, such as about 100, 200, 250, 300, 350 or 400 IU. One international unit (IU) of oxytocin is the equivalent of about 1.67 micrograms of pure peptide. Accordingly, a composition of 1 g of oxytocin gel, 400 IU, is equivalent to about 0.67 mg/g (European Pharmacopoeia 9.2). However, the composition may in other aspects not contain any oxytocin or fragment(s) or variant(s) thereof according to SEQ ID NO:2 (or pharmaceutically acceptable salts of oxytocin or a fragment and/or variant thereof).

Whenever “oxytocin”, “oxytocin peptide” and/or “oxytocin molecule” is referred to herein, this encompasses oxytocin (SEQ ID NO:1) and/or one or more fragment(s) and/or variant(s) thereof as defined herein according to the general formula SEQ ID NO:2, or any other variant and/or fragment as mentioned herein, as well as analogues and/or homologues thereof. Whenever a fragment, variant or homologue of an oxytocin molecule/peptide is envisaged it is to be understood that such a variant, fragment or homologue encompasses a biological activity comparable to the oxytocin molecule itself (SEQ ID NO:1). As an example, it can be shown that a substance has oxytocin activity by performing tests showing the activity of the actual substance, e.g. by performing a double-blind cross-over randomised protocol as described in WO0178758 (Example 1).

SEQ ID NO:2 is in the context of the present document defined as X₁-X₂-X₃-X₄-Asn-Cys-X₅-X₆-X₇-X₈-NH₂
wherein
X₁ is selected from the group consisting of Cys and nothing;
X₂ is selected from the group consisting of Tyr, Phe, and nothing;
X₃ is selected from the group consisting of Ile, Val, Hoph, Phe, Cha, and nothing;
X₄ is selected from the group consisting of Gln, Ser, Thr, Cit, Arg, and Daba;
X₅ is selected from the group consisting of Pro and nothing;
X₆ is selected from the group consisting of Ile, Leu, nothing, Val, Hos, Daba, Thr, Arg, and Cit;
X₇ is selected from the group consisting of Gly, nothing, and Ala;
X₈ is selected from the group consisting of Gly and nothing; with the proviso that SEQ ID NO:2 does not include vasopressin.

The composition disclosed herein may be prepared by mixing the one or more non-ionic cellulose ethers with water and optionally one or more pH regulating agents and/or one or more preservatives.

The composition described herein may e.g. be a composition comprising or consisting of hydroxypropylmethylcellulose, lactic acid and benzoic acid, said composition having a viscosity of about 35 000 cP or more, an osmolality of from about 10 to about 300 mOsmol/kg, such as from about 10 to about 200 mOsmol/kg, from about 20 to about 100 mOsmol/kg, from about 30 to about 50 mOsmol/kg and a pH of from about 3 to about 4. The concentration of lactic acid and benzoic acid and the pH of the composition may be as described elsewhere herein.

The composition described herein may be vaginally administered. Typically, about 0.5-1.5 ml, such as about 1 ml of the composition is administered once daily, although it is possible to administer the composition two or more times a day. The composition is preferably administered when going to bed.

The present document is also directed to a composition as defined herein for use in the treatment and/or prevention of a climacteric disorder, wherein said climacteric disorder is selected from the group consisting of vaginal dryness, vaginal irritation, vaginal itching, dysuria, dyspareunia, and/or vaginal bleeding during and/or after sexual intercourse and any combination thereof.

The present document is also directed to a method for treating and/or preventing a climacteric disorder, said climacteric disorder being selected from the group consisting of vaginal dryness, vaginal irritation, vaginal itching, dysuria, dyspareunia, and/or vaginal bleeding during and/or after sexual intercourse and any combination thereof, wherein said method comprises administration of a pharmaceutically effective amount of a composition as described herein to a subject in need thereof.

The present document is further directed to the use of a non-ionic cellulose ether for the manufacture of a pharmaceutical composition as defined herein for the treatment and/or prevention of a climacteric disorder, wherein said climacteric disorder is a selected from the group consisting of vaginal dryness, vaginal irritation, vaginal itching, dysuria, dyspareunia, and/or vaginal bleeding during and/or after sexual intercourse and any combination thereof.

Also disclosed herein is a kit of parts comprising:

(i) a composition as defined herein

(ii) a dispenser for said composition, and

(iii) optionally instructions for use.

The present document is also directed to hydroxypropylmethylcellulose for use in the treatment and/or prevention of a climacteric disorder selected from the group consisting of vaginal dryness, vaginal irritation, vaginal itching, dysuria, dyspareunia, and/or vaginal bleeding during and/or after sexual intercourse and any combination thereof. The present document is also directed to the use of hydroxypropylmethylcellulose for the manufacture of a pharmaceutical composition for the treatment and/or prevention of a climacteric disorder, wherein said climacteric disorder is a selected from the group consisting of vaginal dryness, vaginal irritation, vaginal itching, dysuria, dyspareunia, and/or vaginal bleeding during and/or after sexual intercourse and any combination thereof. The present document is also directed to a method for treating and/or preventing a climacteric disorder, said climacteric disorder being selected from the group consisting of vaginal dryness, vaginal irritation, vaginal itching, dysuria, dyspareunia, and/or vaginal bleeding during and/or after sexual intercourse and any combination thereof, wherein said method comprises administration of a pharmaceutically effective amount of hydroxypropylmethylcellulose to a subject in need thereof.

The invention will be further described in the following examples, which do not limit the scope of the invention described in the claims.

### EXPERIMENTAL SECTION

### General

The equipment used for mixing was a Unimix SRT 15. The hypromellose used was Benecel K15M Pharm.

### Example 1: Pharmaceutical Composition Manufacturing

The components of Table 1 were mixed as follows. Purified water (1 371 g) was added to a container followed by lactic acid (33 g). Mixing was performed until a homogeneous solution, as indicated by visual inspection, was obtained. The pH of the homogenous solution was measured and found to be 2.72. The pH was adjusted to 3.72 by addition of a 5 M aqueous solution of NaOH. Thereafter, purified water was added (719.3 g) followed by benzoic acid (15 g) at a mixing speed of 4.5 rpm. Homogenization was activated for 125 s at a mixing speed of 4.5 rpm. Mixing was continued for 90 minutes. Then, visual inspection revealed that all benzoic acid was dissolved. The solution was allowed to assume room temperature, and then hypromellose (450 g) was added to the solution. The resulting solution was mixed at about 12° C. at a mixing speed of about 2.5 rpm for 121 minutes. During this time, the homogenizer was activated for about 1 minute. Thereafter, mixing was continued at a mixing speed of about 2.5 rpm at room temperature for 18 hours. The resulting gel was homogenous as shown by visual inspection. No lumps or air bubbles were present.

TABLE 1

| Component | Amount per batch (g) |
|---|---|
| Benzoic acid | 15 |
| Lactic acid | 33 |
| Sodium hydroxide 5M | q.s.* |
| Hypromellose (Benecel) | 450 |
| Purified water | q.s. ** |

*To a pH of 3.75 (q.s. stands for quantum satis)
** To a final weight of 15 000 g

Visual inspection showed that the gel was substantially clear. The viscosity was measured at 20° C. according to European Pharmacopoeia 7.0, 2.2.10 at 1-12 rpm as well as the pH was measured providing values shown in Table 2. The pH was 3.6.

TABLE 2

| Mixing speed in rpm | Viscosity value in cP |
|---|---|
| 1 | 62 500 cP |
| 3 | 50 167 cP |
| 5 | 43 800 cP |
| 10 | 35 100 cP |
| 12 | 32 833 cP |

### Example 2: Storage Stability

The storage stability of the pharmaceutical composition of Example 1 was tested at a temperature of about 2-8° C. when kept in aluminum tubes. The storage stability was monitored by measurement of viscosity and pH as shown in Table 3.

TABLE 3
Viscosity and pH as a function of time after
storage in aluminium tube at 2-8° C.

| | | Viscosity | Viscosity | Viscosity |
|---|---|---|---|---|
| | | at | at | at |
| Analysis | Limits | 0* months | 6 months | 12 months |
| Viscosity at | 1 rpm | 52 000 | 47 000** | 11 000** |
| 1 rpm, cP | | | | |
| pH | 3.4-4.2 | 3.6 | 3.6 | 3.6 |

*Initial results measured after 2 months bulk storage
**Uncertain due to low torque value (<10%) during analysis

As shown in Table 3, the viscosity of the pharmaceutical composition kept in the aluminum tube decreased with time, and in particular after six months' storage (i.e. after 8 months' storage from date of production).

### Example 3: Effect of Composition of Example 1 on the Most Bothersome Symptom

In this clinical study, the participating women were instructed to score their MBS at a scale between 0 and 3, wherein 0 is no symptom of MBS, 1 is mild symptoms, 2 is moderate symptoms, and 3 is severe symptoms of MBS.

A clinical study was performed using the pharmaceutical composition of Example 1. Postmenopausal women with severe and moderate symptoms of vaginal irritation and itching, dyspareunia, vaginal dryness, dysuria or presence of vaginal bleeding associated with sexual intercourse that had been self-identified by the subject as being the most bothersome to her (i.e. the Most Bothersome Symptom, MBS), who meet the inclusion and exclusion criteria. 76 women were enrolled to the study and 72 completed it. Vaginal cytology, vaginal pH, and a self-assessment of most bothersome symptoms were assessed. The treatment consisted of administration of 1 ml of the pharmaceutical composition intravaginally once daily for 12 weeks.

Clinical evaluations were performed at the following time points:
Screening Period (Day −35 to Day 0)
Visit 1 Randomization (Week 0, Day 0)
Visit 2 (Week 4, Day 28±3)
Visit 3 End of Treatment/Early Discontinuation (Week 12, Day 84±5)
Telephone Follow-up (Week 14, Day 98±5)* *Study subjects were followed-up by telephone

MBS was scored between 0 and 3 (wherein 0 is no symptoms of MBS, 1 is mild symptoms, 2 is moderate symptoms, and 3 is severe symptoms of MBS) by the women and the MBS values in Tables 4 and 5 are the mean values of the women's' individual scores. The data in Table 4 is the mean of the scores of 45 women, while the data in Table 5 is the mean of the scores of 27 women.

TABLE 4
Effect on most bothersome symptom (MBS) of the pharmaceutical
composition used before 6 months storage of the gel,
i.e. having a viscosity of ≥47000 cP.

| MBS mean | MBS mean | Decrease in MBS score |
|---|---|---|
| score 0 weeks | score 12 weeks | between 0 and 12 weeks |
| 2.61 | 1.24 | −1.37 |

TABLE 5
Effect on most bothersome symptom (MBS) of pharmaceutical
composition used after 6 months storage, i.e.
having a viscosity of <47000 cP

| MBS mean | MBS mean | Decrease in MBS score |
|---|---|---|
| score 0 weeks | score 12 weeks | between 0 and 12 weeks |
| 2.52 | 1.59 | −0.93 |

As can be seen from Tables 3-5 above, the difference in MBS between 0 and 12 weeks was −1.37 when a gel with a high viscosity was used while it was only −0.93 when a gel with a lower viscosity was used. Thus, the viscosity of the gel is important for the gel's effect on MBS.

### Example 4: Effect of Composition of Example 1 on the Most Bothersome Symptom

The pharmaceutical composition (VagiVital™) was prepared in the same way as described in Example 1 and with the same final concentrations of the constituents with the exception for HPMC which was added in an amount resulting in a final concentration of HPMC of 3.2 wt % instead of 3 wt %.

The patients were instructed to administer 1 ml of the composition intravaginally once daily for 12 weeks. The composition was kept refrigerated throughout the study. In the main part of the study, the composition was stored in a pre-filled 1 ml glass syringe while in the exploratory part the composition was stored in a laminate tube from which the patients filled 1 ml in an applicator before administration.

Primary Efficacy Endpoint (pp Analysis Set)

The primary objective of this report is to evaluate the efficacy of VagiVital™ in reducing the severity of the Most Bothersome Symptom (MBS) of vulvovaginal atrophy (VVA) associated with menopause after 12 weeks of treatment.

The primary efficacy endpoint is the change from baseline (V0) to 12 weeks post baseline (V3) in severity of the VVA symptom that has been self-identified by the patient as being the MBS to her at baseline.

TABLE 6
Main Study: MBS (Most Bothersome VVA Symptom identified
at baseline and followed during the study period).
Per Protocol Subset of patients.

| Statistics | V0 | V2 | V3 | V2 − V0 | V3 − V0 |
|---|---|---|---|---|---|
| N | 77 | 77 | 77 | 77 | 77 |
| Missing | 0 | 0 | 0 | 0 | 0 |
| Min | 2 | 0 | 0 | −3 | −3 |
| Median | 2.00 | 1.00 | 1.00 | −1.00 | −1.00 |
| Max | 3 | 3 | 3 | 1 | 1 |
| Mean | 2.45 | 1.47 | 1.18 | −0.99 | −1.27 |
| Std | 0.50 | 0.99 | 1.07 | 0.91 | 1.00 |
| P-value¹ | NA | NA | NA | 0.0000 | 0.0000 |

¹Wilcoxon signed rank test. 2-sided

TABLE 7
Exploratory part: Most Bothersome Symptom.
Per Protocol Subset of patients.

| Statistics | V0 | V2 | V3 | V2 − V0 | V3 − V0 |
|---|---|---|---|---|---|
| N | 9 | 9 | 9 | 9 | 9 |
| Missing | 0 | 0 | 0 | 0 | 0 |
| Min | 2.00 | 0.00 | 0.00 | −3.00 | −3.00 |
| Median | 3.00 | 1.00 | 0.00 | −2.00 | −2.00 |
| Max | 3.00 | 2.00 | 2.00 | −1.00 | −1.00 |
| Mean | 2.56 | 0.67 | 0.56 | −1.89 | −2.00 |
| Std | 0.53 | 0.71 | 0.73 | 0.78 | 0.87 |
| P-value² | NA | NA | NA | 0.0039 | 0.0039 |

²Wilcoxon signed rank test. 2-sided

TABLE 8
Main Study: MBS (Most Bothersome VVA Symptom identified
at baseline and followed during the study period). Shift
(from baseline) table. Number of patients in each severity
category. Per Protocol Subset of patients.

| | Severity | V0 (Baseline) | | | | |
|---|---|---|---|---|---|---|
| Visit | category | None | Mild | Moderate | Severe | Total |
| V2 | None | 0 | 0 | 12 | 2 | 14 |
| | Mild | 0 | 0 | 16 | 7 | 23 |
| | Moderate | 0 | 0 | 12 | 12 | 24 |
| | Severe | 0 | 0 | 2 | 11 | 13 |
| | Missing | 0 | 0 | 1 | 3 | 4 |
| | Total | 0 | 0 | 43 | 35 | 78 |
| V3 | None | 0 | 0 | 19 | 6 | 25 |
| | Mild | 0 | 0 | 17 | 12 | 29 |
| | Moderate | 0 | 0 | 4 | 6 | 10 |
| | Severe | 0 | 0 | 3 | 11 | 14 |
| | Missing | 0 | 0 | 0 | 0 | 0 |
| | Total | 0 | 0 | 43 | 35 | 78 |

TABLE 9
Exploratory part: MBS (Most Bothersome VVA Symptom identified
at baseline and followed during the study period). Shift
(from baseline) table. Number of patients in each severity
category. Per Protocol Subset of patients.

| | Severity | V0 (Baseline) | | | | |
|---|---|---|---|---|---|---|
| Visit | category | None | Mild | Moderate | Severe | Total |
| V2 | None | 0 | 0 | 2 | 2 | 4 |
| | Mild | 0 | 0 | 2 | 2 | 4 |
| | Moderate | 0 | 0 | 0 | 1 | 1 |
| | Severe | 0 | 0 | 0 | 0 | 0 |
| | Missing | 0 | 0 | 0 | 0 | 0 |
| | Total | 0 | 0 | 4 | 5 | 9 |
| V3 | None | 0 | 0 | 2 | 3 | 5 |
| | Mild | 0 | 0 | 2 | 1 | 3 |
| | Moderate | 0 | 0 | 0 | 1 | 1 |
| | Severe | 0 | 0 | 0 | 0 | 0 |
| | Missing | 0 | 0 | 0 | 0 | 0 |
| | Total | 0 | 0 | 4 | 5 | 9 |

In the main study (Table 8) a total of 14 (14/74=19%) patients do not have any symptoms on their most bothersome symptom at V2. The corresponding figure at V3 is 25 (25/78=32%).

In the main study (Table 8) a total of 49 patients ((12+16+2+7+12)/74=66%) have less severe symptoms at V2 compared to baseline. The corresponding figure at V3 is 60 ((19+17+6+12+6)/78=77%).

In the exploratory part (Table 9) a total of 4 (4/9=44%) patients do not have any symptoms on their most bothersome symptom at V2. The corresponding figure at V3 is 5 (5/9=56%).

In the exploratory part (Table 9Table) all 9 patients ((2+2+2+2+1)/9=100%) have less severe symptoms at V2 compared to baseline. The corresponding figure at V3 is as well 9 ((2+2+3+1+1)/9=100%).

Secondary Efficacy Endpoints (pp Analysis Set)

Change from baseline (V0) until 4 (V2) and 12 (V3) weeks post baseline in vaginal pH (decrease is positive).

TABLE 10
Main Study: pH. Per Protocol Subset of patients.

| Statistics | V0 | V2 | V3 | V2 − V0 | V3 − V0 |
|---|---|---|---|---|---|
| N | 77 | 77 | 77 | 77 | 77 |
| Missing | 0 | 0 | 0 | 0 | 0 |
| Min | 5 | 4 | 4 | −4 | −3 |
| Median | 7.40 | 6.70 | 6.40 | −0.40 | −0.50 |
| Max | 9 | 9 | 8 | 1 | 1 |
| Mean | 7.00 | 6.48 | 6.28 | −0.52 | −0.72 |
| Std | 0.94 | 1.36 | 1.33 | 1.02 | 1.09 |
| P-value³ | NA | NA | NA | 0.0001 | 0.0000 |

³Wilcoxon signed rank test. 2-sided

TABLE 11
Exploratory part: pH. Per Protocol Subset of patients.

| Statistics | V0 | V2 | V3 | V2 − V0 | V3 − V0 |
|---|---|---|---|---|---|
| N | 9 | 9 | 9 | 9 | 9 |
| Missing | 0 | 0 | 0 | 0 | 0 |
| Min | 5.20 | 4.30 | 4.20 | −1.30 | −2.60 |
| Median | 6.30 | 6.60 | 5.90 | −0.10 | −0.50 |
| Max | 8.00 | 8.30 | 8.10 | 2.00 | 1.80 |
| Mean | 6.67 | 6.61 | 6.10 | −0.06 | −0.57 |
| Std | 1.16 | 1.47 | 1.47 | 1.00 | 1.32 |
| P-value⁴ | NA | NA | NA | 0.7148 | 0.2344 |

⁴Wilcoxon signed rank test. 2-sided

Change from baseline (V0) until 4 (V2) and 12 (V3) weeks post baseline in Percent superficial cells (increase is positive).

TABLE 12
Main Study: Superficial cells. Per Protocol Subset of patients.

| Statistics | V0 | V2 | V3 | V2 − V0 | V3 − V0 |
|---|---|---|---|---|---|
| N | 77 | 76 | 77 | 76 | 77 |
| Missing | 0 | 1 | 0 | 1 | 0 |
| Min | 0 | 0 | 0 | −4 | −4 |
| Median | 0.00 | 0.00 | 0.00 | 0.00 | 0.00 |
| Max | 5 | 60 | 26 | 55 | 26 |
| Mean | 0.45 | 2.86 | 2.42 | 2.42 | 1.96 |
| Std | 1.16 | 8.90 | 5.44 | 8.32 | 5.15 |
| P-value⁵ | NA | NA | NA | 0.0011 | 0.0003 |

⁵Wilcoxon signed rank test. 2-sided

TABLE 13
Exploratory Part: Superficial. Per Protocol Subset of patients.

| Statistics | V0 | V2 | V3 | V2 − V0 | V3 − V0 |
|---|---|---|---|---|---|
| N | 9 | 9 | 9 | 9 | 9 |
| Missing | 0 | 0 | 0 | 0 | 0 |
| Min | 0.00 | 0.00 | 0.00 | 0.00 | 0.00 |
| Median | 0.00 | 0.00 | 0.00 | 0.00 | 0.00 |
| Max | 2.00 | 7.00 | 18.00 | 5.00 | 16.00 |
| Mean | 0.22 | 1.33 | 2.89 | 1.11 | 2.67 |
| Std | 0.67 | 2.69 | 5.90 | 2.20 | 5.27 |
| P-value⁶ | NA | NA | NA | 0.5000 | 0.1250 |

⁶Wilcoxon signed rank test. 2-sided

### Summary of Results

Efficacy

Main Part of the Study
Primary efficacy endpoint:
   There was a statistically significant improvement from baseline until both 4 weeks post baseline and 12 weeks post baseline
   32% of the patients do not have any symptoms on their most bothersome 12 weeks post baseline
   77% of the patients have less severe symptoms 12 weeks post baseline compared to baseline
Secondary efficacy endpoints
   pH
      There was a statistically significant improvement from baseline until both 4 weeks post baseline and 12 weeks post baseline
   Superficial cells
      There was a statistically significant improvement from baseline until both 4 weeks post baseline and 12 weeks post baseline
   Vaginal dryness
      There was a statistically significant improvement from baseline until both 4 weeks post baseline and 12 weeks post baseline
   Vaginal/vulvar irritating/itching
      There was a statistically significant improvement from baseline until both 4 weeks post baseline and 12 weeks post baseline
   Pain, burning or stinging during urination
      There was a statistically significant improvement from baseline until both 4 weeks post baseline and 12 weeks post baseline
   Vaginal discomfort and/or pain associated with vaginal sexual activity
      There was a statistically significant improvement from baseline until both 4 weeks post baseline and 12 weeks post baseline
   Parabasal cells and Maturation value
      There was not a statistically significant change from baseline until neither 4 weeks post baseline nor 12 weeks post baseline
   Quality of Life
      [symbol] [symbol] the patients were asked about (urgency) urinary incontinence and the results are clearly indicating an improvement over time.

Exploratory Part of the Study

The statistical power is low as only 9 patients are included in the efficacy analyses and this should be taken into consideration when valuing statistical significances.
Primary efficacy endpoint:
   There was a statistically significant improvement from baseline until both 4 weeks post baseline and 12 weeks post baseline.
   The improvement from baseline was numerically superior to the improvement in the main part of the study which indicates that the laminate tube (used in the exploratory part) was at least as well accepted as the glass syringes (used in the main part of the study)
   56% of the patients do not have any symptoms on their most bothersome 12 weeks post baseline
   100% of the patients have less severe symptoms 12 weeks post baseline compared to baseline [symbol]
Secondary efficacy endpoints
   pH
      There was a numerically but not statistically significant improvement from baseline until both 4 weeks post baseline and 12 weeks post baseline
   Superficial cells
      There was a numerically but not statistically significant improvement from baseline until both 4 weeks post baseline and 12 weeks post baseline
   Vaginal dryness
      There was a statistically significant improvement from baseline until both 4 weeks post baseline and 12 weeks post baseline
   Vaginal/vulvar irritating/itching
      There was a statistically significant improvement from baseline until both 4 weeks post baseline and 12 weeks post baseline
   Pain, burning or stinging during urination
      There was a numerically but not statistically significant improvement from baseline until both 4 weeks post baseline and 12 weeks post baseline
   Vaginal discomfort and/or pain associated with vaginal sexual activity
      There was a statistically significant improvement from baseline until both 4 weeks post baseline and 12 weeks post baseline
   Parabasal cells
      There was not a statistically significant change from baseline until 4 weeks post baseline but here was a statistically significant change until 12 weeks post baseline
   Maturation value
      There was not a statistically significant change from baseline until 4 weeks post baseline but here was a statistically significant change until 12 weeks post baseline
   Quality of Life
      [symbol] [symbol] the patients were asked about (urgency) urinary incontinence and the results are clearly indicating an improvement over time.

### OVERALL CONCLUSIONS

Patients using VagiVital™ reported a significant reduction in the severity of the most bothersome VVA symptom as well as improved (decreased) vaginal pH and increased percentage superficial cells over a 12-week treatment period
The magnitude of the effect of VagiVital™ on MBS is on the same level as has been reported for oestrogen based products (e.g. Vagifem® (estradiol vaginal inserts))
The improvement regarding urgency urinary incontinence is of high importance for the patients and offers an additional benefit of VagiVital™.
There were no safety or tolerability concerns associated with VagiVital™

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Unless expressly described to the contrary, each of the preferred features described herein can be used in combination with any and all of the other herein described preferred features.

1. A composition for use in the treatment and/or prevention of a climacteric disorder, wherein said climacteric disorder is selected from the group consisting of vaginal dryness, vaginal irritation, vaginal itching, dysuria, dyspaereunia, and/or vaginal bleeding during and/or after sexual intercourse and any combination thereof, said composition comprising at least one non-ionic cellulose ether, wherein said composition has a viscosity of 35000 mPa-S or more, said viscosity being measured at 20° C. according to European Pharmacopoeia 7.0, 2.2.10, an osmolality of from about 10 to about 300 mOsmol/kg, and a pH of from 3 to 4, and wherein said composition does not comprise an active pharmaceutical agent.

2. A method according to claim 10, wherein said composition has a viscosity of at least 55 000 mPa-s.

3. A method according to claim 10, wherein said composition has a viscosity of from 35 000 to 100 000 mPa-s.

4. A method according to claim 10, wherein the osmolality of said composition is from 10 to 200 mOsmol/kg.

5. A method according to claim 10, wherein said composition has a pH within the range of from 3 to 3.8.

6. A method according to claim 10, wherein the pH of said composition is regulated by adding a pH regulating agent to said composition.

7. A method according to claim 10, said composition further comprising a preservative.

8. A method according to claim 10, wherein said at least one non-ionic cellulose ether is selected from the group consisting of methylcellulose (MC), hydroxypropylcellulose (HPC), hydroxypropylmethylcellulose (HPMC), hydroxyethylethylcellulose (HEEC) and hydroxyethylmethylcellulose (HEMC) and any combination thereof.

9. A method according to claim 10, wherein said at least one non-ionic cellulose ether is hydroxypropylmethylcellulose (HPMC).

10. A method for treating and/or preventing a climacteric disorder, said climacteric disorder being selected from the group consisting of vaginal dryness, vaginal irritation, vaginal itching, dysuria, dyspareunia, and/or vaginal bleeding during and/or after sexual intercourse and any combination thereof, wherein said method comprises administering to a subject in need thereof an amount which is effective to treat said climacteric disorder of a composition which does not comprise an active pharmaceutical agent, said composition comprising at least one non-ionic cellulose ether, wherein said composition has a viscosity of 35000 mPa-S or more, said viscosity being measured at 20° C. according to European Pharmacopoeia 7.0, 2.2.10, an osmolality of from about 10 to about 300 mOsmol/kg, and a pH of from 3 to 4, and wherein the composition is free of oxytocin.

11. A method according to claim 10, wherein said composition has a viscosity of from 55 000 to 100 000 mPa-s.

12. A method according to claim 10, wherein said composition after storage at room temperature for about six months has a viscosity of at least 38 000 mPa-s.

13. A method according to claim 10, wherein said composition after storage at room temperature for about six months has a viscosity of at least 55 000 mPa-s.

14. A method according to claim 10, wherein the osmolality of said composition is from about 30 to about 50 mOsmol/kg.

15. A method according to claim 10, wherein said composition has a pH within the range of about 3 to 3.3.

16. A method according to claim 6, wherein the pH regulating agent is a buffer.

17. A method according to claim 16, wherein the buffer is a lactate buffer or a citrate buffer.

18. A method according to claim 7, wherein the preservative is benzoic acid.
